(19) United States
(12) Patent Application Publication
(10) Pub. No.: US 2005/0282892 A1
(43) Pub. Date: Dec. 22, 2005

(54) Pharmaceutical composition useful for treating chronic myeloid leukemia

(76) Inventors: Santu Bandyopadhyay, Kolkata (IN); Bikas Chandra Pal, Kolkata (IN); Samir Bhattacharyay, Kolkata (IN); Swapan Mondal, Kolkata (IN); Chhabinath Mandal, Kolkata (IN); Aditya Konar, Kolkata (IN); Keshab Chandra Roy, Kolkata (IN); Tanusree Biswas, Kolkata (IN); Gautam Bandyopadhyay, Kolkata (IN)

Correspondence Address: FITZPATRICK CELLA HARPER & SCINTO, 30 ROCKEFELLER PLAZA, NEW YORK, NY, 10112, US

(21) Appl. No.: 11/174,545

(22) Filed: Jul. 6, 2005

Related U.S. Application Data
(63) Continuation-in-part of application No. 10/338,689, filed on Jan. 9, 2003, now abandoned.
(60) Provisional application No. 60/393,750, filed on Jul. 8, 2002.

(51) Int. Cl. A61K 31/235

(52) U.S. Cl. 514/533

(57) ABSTRACT

This invention relates to a pharmaceutical composition useful for treating chronic myeloid leukemia where Bcr-Abl kinase is constitutively expressed in animals and humans, and a treatment of chronic myeloid leukemia (CML) by a composition comprising an effective amount of analogs and/or salts of chlorogenic acid. The analogs are preferably sodium chlorogenate (Na-Chl) or potassium or ammonium salts, which were prepared from Chlorogenic acid or its analogs.

This application is a Continuation-In-Part of application Ser. No. 10/338,689, filed on Jan. 9, 2003, which claims benefit of U.S. Provisional App. No. 60/393,750 filed on Jul. 8, 2002.

### FIELD OF INVENTION

This invention relates to the treatment of chronic myeloid leukemia (CML) by a composition comprising analogs and/or salts of chlorogenic acid, salts represented by formula 1 wherein R1 repesents —OH or S1 or S3; R2 represents —OH or S1; R3 represents S2 or —OH ans salts of analogs of chlorogenic acid. Wherein, the analogs are preferably sodium chlorogenate (Na-Chl) or potassium or ammonium salts, or any other salts prepared from Chlorogenic acid or its analogs.

Chronic myeloid leukemia is lethal, there is no drug directed towards the destruction of the leukemic cells, and these cells poorly respond to chemotherapy which is always non-specific thus adversely affecting normal cells. Unique property of the therapy with NaChl is the killing of myeloid cancer cells leaving other normal cells unaffected.

### BACKGROUND AND PRIOR ART DESCRIPTION

Myeloid leukemia is usually subdivided into two groups: Acute Myeloid Leukemia (AML) and Chronic Myeloid Leukemia (CML). AML is characterized by an increase in the number of myeloid cells in the bone marrow and an arrest in their maturation. In the United States, the annual incidence of AML is approximately 2.4 per 100,000 and it increases progressively with age, to a peak of 12.6 per 100,000 adults 65 years of age or older. The CML is a malignant clonal disorder of hematopoietic stem cells. The median age at presentation is 53 years, but it occurs at all age groups, including children. The natural history of CML is progression from a benign chronic phase to a rapidly fatal blast crisis within three to five years or even earlier. The prognosis of CML is also poor in spite of vast advancement of clinical medicine. See, Reference (1). CD33 represents a specific and useful marker in the process of myeloid cell differentiation See, Reference (2). Recent reports suggest that engagement of CD33 by monoclonal antibody induced apoptosis leading to growth inhibition of proliferation of AML and CML cells in vitro (See, References (2,3). Exploiting the myeloid specific expression of CD33, humanized anti-CD33 monoclonal antibody conjugated with anti-cancer drug has been tried in AML patients with significant success See, Reference (4). Similarly, lymphoid leukemia is also subdivided in two groups: acute lymphocytic leukemia (ALL) and chronic lymphocytic leukemia (CLL). Lymphoid leukemia may affect both T and B cell lineages and are prevalent in children. Extracts from Piper betel leaves with some anti-myeloid activity were disclosed in PCT/INOO/00118 dated Dec. 12, 2000. Two compounds isolated from Piper betel extract that showed anti myeloid leukemic activity are 3-O-Coumaryl quinic acid, disclosed in copending U.S. application Ser. No. 10/448,398, filed May 30, 2003 and Chlorogenic acid, disclosed in copending U.S. application Ser. No. 10/613,122, filed Jul. 7, 2003.

The present application likewise involves anti myeloid leukemic activity but teaches sodium chlorogenate prepared from the fractions of the betel leaf extracts for treating chronic myeloid leukemia. Chlorogenic acid (Chl) is known to have anti-allergic activity See, Reference (5). Chl also is known to inhibit hepatic and renal glucose-6-phosphatase systems See, Reference (6). Chl is known as an inhibitor of epidermal lypoxygenase activity and TPA-induced ear inflammation See, Reference (7). Chl also is known to render inhibitory effects on TPA-induced tumor promotion in mouse skin See, Reference (7). Certain anti-HIV activity of Chl also has been reported See, Reference (8). The inadvertent fusion of Bcr with the Abl gene results in a constitutively active tyrosine kinase (Bcr-Abl) that transforms cells to chronic myelogenous leukemia (CML) See, Reference (9). Highly potent small molecules are known to inhibit Bcr-Abl dependent cell growth. See, References (10, 11). Recent reports on the development of resistance to one such compound emphasizes the need for further therapeutic search to control CML. See, Reference (12).

The present invention involves a chemical compound isolated from a herb, Piper betel which inhibits Abl protein tyrosine kinase triggering apoptosis of Bcr-Abl and the expressing of CML cell line K562. Elucidation of structure identifies this molecule as chlorogenic acid. Its sodium, potassium, ammonium and other salts also exhibit killing activity against CML cells although the sodium salt, sodium chlorogenate (NaChl) exhibits more potency than other salts of chlorogenic acid. Sodium chlorogenate shows 2.0 fold greater efficiency in killing K562 cells compared to chlorogenic acid. Interestingly, NaChl also destroys Bcr-Abl expressing peripheral blood cells of CML patients without any effects on peripheral blood cells of Bcr-Abl negative CML patients. Analysis of molecular models indicates that Na-Chl occupies the ATP-binding site of the kinase domain of Abl. NaChl therefore is taught herein as an additional therapeutic agent for CML. More particularly, an anti myeloid leukemic activity of Na-Chl is identified herein for the first time.

Chien et al. teach a taste-modifying effect of chlorogenic acid in U.S. patent Pub. Ser. No. 2004-0213881-A1 but neither disclose nor suggest any anti-leukemic activity of chlorogenic acid. The prior art represented by Chien et al. does not, in any way, relate to the present application. The present application discloses in vitro experiments with chlorogenic acid and sodium chlorogenate on Bcr-Abl⁺ CML cell lines, primary cells from CML patients. Data on in vivo xenograft experiments with Bcr-Abl⁺ human CML cell line K562 in nude mice also has been published (See, the article by Bandyopadhyay G, Biswas T, Roy K C, Mandal S, Mandal C, Pal B C, Bhattacharya S, Rakshit S, Bhattacharya D K, Chaudhuri U, Konar A and Bandyopadhyay, entitled “S. Chlorogenic acid inhibits Bcr-Abl tyrosine kinase and triggers p38 mitogen-activated protein kinase-dependent apoptosis in chronic myelogenous leukemic cells”, Blood, 104: 2514-2522, 2004). Data included herein as example 11 and FIG. 5, indicate that salts of chlorogenic acid may be useful for CML patients. Similar approaches have previously been reported for other molecules, including one of which is marketed for the treatment of CML. (See, e.g., Golas, J. M. et al. Cancer Research, 63: 375-381, 2003; Attoub, S. et al. Cancer Research, 62: 4879-4883, 2002).

Disclosures involving in vitro data with sodium chlorogenate on ST1571-resistant K562 cells (K562-R), are included herein as example 12 and FIG. 6, which indicate that salts of chlorogenic acid may be useful for relapse CML patients and Gleevec-resistant CML patients. It has been reported that relapse of CML patients receiving Gleevec is usually due to development of a resistance to Gleevec [See, Barthe, C. et al. Science, 293: 2163a, 2001 (Technical Comments)].

Graus et al. (U.S. Pat. No. 6,632,459) merely suggests one intended use of chlorogenic acid could be for immune system stimulation and may include the treatment of cancer, but without any enabling teachings or specific data. It is well established in the literature that there are hundreds of type of cancers and each cancer condition is a reflection of different causative factors and different cellular behaviors; one compound capable of treating one type of cancer is unlikely to be useful in treating other types of cancer. Therefore, any suggestions within Graus et al as to possible uses of chlorogenic acid for the treatment of virtually everything i.e. cancer, a toxin, infection by a parasite, a virus and/or a bacterium is non-enabling in the extreme. The present patent application includes specific data on sodium chlorogenate and anti-leukemic activity. Further, Graus et al also suggested that chlorogenic acid might stimulate T helper cells to develop particular cytokines which support other types of lymphocytes. Examples of such lymphocyte supporting cytokines are interleukin-2 (IL-2), IL-6 and interferon-gamma (IFN-γ). However, in the present application data is disclosed, as in example 13 and Table II, which indicates how chlorogenic acid, in fact, suppresses the production of such cytokines. Therefore, the prior art represented by Graus et al does not relate to the present application.

U.S. patent Pub. Ser. No. 2003-0229140-A1 (hereafter “Bandyopadhyay et al. 140”) discloses the use of chlorogenic acid or 3-O-p-coumaryl quinic acid alone, or in combination, for an anti-leukemic activity. In contrast, the present application discloses novel anti-leukemic activity of salts of chlorogenic acid and further defines four analogs of chlorogenic acid and their salts. These defined analogs of chlorogenic acid taught only in the present application are Neochlorogenic acid, Cryptochlorogenic acid, 3-O-(3′-methyl caffeoyl) quinic acid and 5-O-(caffeoyl-4′-methyl) quinic acid. Neither 3-O-p-coumaryl quinic acid nor chlorogenic acid are specified in the present application. Therefore, the prior art represented by Bandyopadhyay et al. 140 does not relate to the present application.

N'Guyen, Quang-Lan (U.S. Pat. No. 5,686,062) and Cragoe et al (U.S. Pat. No. 3,966,966) both discuss how a a salt form of a parent compound can be used for the same purpose as the parent compound, but neither document teaches why or how a salt might be more effective than a parent compound. In the FIG. 2d of the present application and also in the above-noted publication using some common data (Blood 104: 2514-2522; 2004) it is taught that sodium chlorogenate is twice as potent as chlorogenic acid in killing myeloid leukemia cancer cell line K562 in vitro. Thus, the present application involves teachings about salts of certain compounds that are more effective than its parent compound. Therefore, the prior art represented by N'Guyen, Quang-Lan and Cragoe et al do not relate to the present application.

PCT/IN00/00118 (hereafter “Bandyopadhyay et al. 118”) discloses an antileukemic activity of crude Piper betel leaf extract. The crude Piper betel leaf extract contains thousands of different molecules and the anti-monocytic activity is unlikely to be associated with chlorogenic acid or its salts. Note that FIG. 1 of Bandyopadhyay et al. 118 teaches that normal human peripheral blood monocytes specifically are destroyed by crude Piper betel leaf extract. On the other hand, in FIG. 3a of the present application and also in the above-noted publication using some common data (Blood 104: 2514-2522; 2004) it is specifically taught that NPBMC (normal human peripheral blood mononuclear cells which contain significant proportion of monocytes) remain virtually unaffected by sodium chlorogenate. Therefore, the prior art represented by Bandyopadhyay et al. 118 does not relate to the present application.

The prior art represented by co-pending application Ser. No. 10/319,618 (U.S. Pat. Pub. Ser. No. 2003-0108632-A1, published Jun. 12, 2003) discloses Th1 type immune response by crude Piper betel leaf extract where it was shown that crude Piper betel leaf extract induced the production of interferon-gamma while at the same time reduced the production of interleukin-4 both at the level of mRNA (FIG.-1) and proteins (FIG.-2). In contrast, the present patent application does not deal with Th1 type immune response; rather it discloses anti-leukemic activity of four defined analogs of chlorogenic acid. Further, data disclosed herein as Table II clearly indicate that sodium chlorogenate significantly inhibits the production of interferon gamma, thereby further illustrating that the active principles underlying U.S. patent Pub. Ser. No. 2003-0108632-A1 and the present application are separate and distinct. Therefore, the prior art represented by U.S. patent Pub. Ser. No. 2003-0108632-A1 does not relate to the present application.

The copending application Ser. Nos. 10/207,039 (now U.S. Pat. No. 6,852,344-B1, issued Feb. 8, 2005); 10/613,122 (U.S. patent Pub. Ser. No. 2004-0052874-A1); and 10/338,688 (U.S. patent Pub. Ser. No. 2003-0229140-A1); each disclose the anti-leukemic activity of chlorogenic acid and 3-O-p-coumaryl quinic acid either alone or in combination. However, none of these commonly-owned applications deal with salts of chlorogenic acid or the four analogs of chlorogenic acid as disclosed in the present patent application. Additionally, sodium salt of chlorogenic acid has been shown in the present application to be twice as potent as the chlorogenic acid in killing chronic myeloid leukemic cell line K562. Therefore, these copending patent applications do not relate to the present application.

### REFERNCES

1. Sawyers C L, The New England Journal of Medicine, 340 (17): 1330-1340, 1999.

2. Vitale C, Romagnani C et. al. Proc. Natl. Acd. Sci. USA, 96 (26): 15091-15096, 1999.

3. Vitale C et. al. Proc. Natl. Acd. Sci, USA., 98 (10): 5764-5769, 2001.

4. Sievers E L, Appelbaum F R et. al. Blood, 93: 3678-3684, 1999.

5. Ito H, Miyazaki T, Ono M and Sakurai H. Bioorg. Med. Chem. 6(7): 1051-1056, 1998.

6. Arion W J et. al. Arch. Biochem. Biophys. 351(2): 279-285, 1998.

7. Conney A H et. al. Adv. Enzyme Regul. 31: 385-396, 1991.

8. Supriyatna G et. al. Phytomedicine, 7 (Suppl. II): 87, 2000.

9. Rowley J D. Nature 243: 290-293, 1973.

10. Druker B J et. al. Nature Medicine 2: 561-566, 1996.

11. Nagar B et. al. Cancer Research 62: 4236-4243, 2002.

12. Coutre P L et. al. Blood 95: 1758-1766, 2000.

### OBJECTS OF THE INVENTION

The main object of the invention is to provide a pharmaceutical composition comprising analogs and/or salts of chlorogenic acid and/or its salts represented by formula 1 and salts of analogs of chlorogenic acid.

Another object of the present invention is to provide a pharmaceutical composition comprising analogs and/or salts of chlorogenic acid and/or its salts represented by formula 1 and salts of analogs of chlorogenic acid 1 for treating chronic myeloid leukemia.

Another object of the invention is to provide pharmaceutical composition, comprising analogs of salts of chlorogenic acid such as sodium chlorogenate (Na-Chl) or potassium or ammonium salts, which were prepared from Chlorogenic acid or its analogs, for the treatment of chronic myeloid leukemia.

Another object of the invention is to provide a new use of the compound sodium chlorogenate (NaChl) prepared from Chlorogenic acid (Chl) isolated from the Piper betel leaf extract or from any other sources for the treatment of chronic myeloid leukemia.

Another objective of the invention is to provide a new pharmaceutical composition comprising a carrier along with the compound sodium chlorogenate for the treatment of chronic myeloid leukemia.

Yet another objective of the invention is to provide a process for the preparation of sodium chlorogenate from Chlorogenic acid to treat CML.

Yet another objective of the invention is to provide a simplified method of preparation of NaChl from Chlorogenic acid which was isolated from all plant parts of Piper betel possessing biological activities relevant to the treatment of CML.

Yet another objective of the invention is to provide sodium salt of Chlorogenic acid a herbal product from leaves or any other plant parts of Piper betel for the treatment of CML.

### SUMMARY OF THE INVENTION

Accordingly, the present provides a pharmaceutical composition for the treatment of chronic myeloid leukemia (CML) by a composition comprising analogs and/or salts of chlorogenic acid and salts of analogs of chlorogenic acid represented by Formual 1, such as sodium chlorogenate (Na-Chl) or potassium or ammonium salts, which were prepared from Chlorogenic acid or its analogs or salts or salts of analogs of chlorogenic acid.

In particular, the present invention provides a pharmaceutical composition, which kills myeloid cancer cells leaving other normal cells unaffected. Chronic myeloid leukemia is lethal, there is no drug directing towards the destruction of the leukemic cells, and these cells poorly respond to chemotherapy which is always non-specific thus adversely affecting normal cells.

Unique property of the therapy with analogs and/or salts of chlorogenic acid is the killing of myeloid cancer cells leaving other normal cells unaffected.

### DETAILED DESCRIPTION OF THE INVENTION

Accordingly, the present invention provides a pharmaceutical composition useful for treating chronic myeloid leukemia where Bcr-Abl kinase is constitutively expressed in animals and humans, said composition comprising an effective amount of analogs and/or salts of chlorogenic acid and salts of analogs of chlorogenic acid represented by formula 1, wherein R1 repesents —OH or S1 or S3; R2 represents —OH or S1; R3 represents S2 or —OH. along with pharmaceutically acceptable additives. The group of such analogs of chlorogenic acid have the following chemical structure.

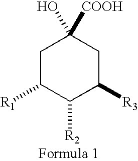

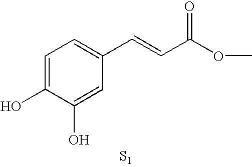

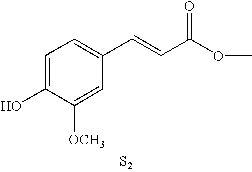

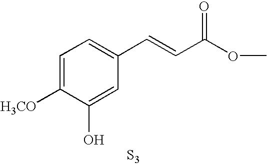

| | Identity of | Identity of | Identity of |
|---|---|---|---|
| Compound | R₁ | R₂ | R₃ |
| Neochlorogenic acid | S₁ | OH | OH |
| Cryptochlorogenic acid | OH | S₁ | OH |
| 3-O-(3′-methyl caffenoyl) quinic acid | OH | OH | S₂ |
| 5-O-(caffenoyl-4′-methyl) quinic acid | S₃ | OH | OH |

Another embodiment of the present invention provides a method of treating chronic myeloid leukemia CML where Bcr-Abl kinase is constitutively expressed in animals and humans, said method comprising administering a pharmaceutical composition comprising an effective amount of analogs and/or salts of chlorogenic acid represented in formula 1, wherein R1 repesents —OH or S1 or S3; R2 represents —OH or S1; R3 represents S2 or —OH. and salts of analogs of chlorogenic acid represented by formula 1 along with pharmaceutically acceptable additives.

Another embodiment provides a use of pharmaceutical composition as claimed in claim 1 for the treatment of chronic myeloid leukemia in a subject where Bcr-Abl kinase is constitutively expressed in animals and humans, said composition comprising an effective amount of analogs and/or salts of chlorogenic acid and represented by formula 1 and salts of analogs of chlorogenic acid represented by formula 1 along with pharmaceutically acceptable additives.

In an embodiment of the invention, the said composition is also useful in treating diseases caused by over expression of Abl type of kinase.

In another embodiment, the analogs of chlorogenic is selected from a group consisting of sodium chlorogenate (FIG. 1), neochlorogenic acid (5-O-caffeoyl quinic acid), cryptochlorogenic acid (4-O-Caffeoyl quinic acid), 3-O-(3′-methylcaffeoyl) quinic acid and 5-O-(Caffeoyl-4′-methyl) quinic acid.

In still another embodiment, the analogs and/or salts of chlorogenic acid and salts of analogs of chlorogenic acid are obtained either from natural sources or synthetically prepared.

In still another embodiment, the salt of chlorogenic acid analog is selected from sodium, potassium and ammonium.

In yet another embodiment, the additive is selected from a group consisting of nutrients such as proteins, carbohydrates, sugars, talc, magnesium stearate, cellulose, calcium carbonate, starch-gelatin paste and/or pharmaceutically acceptable carriers, excipient, diluents or solvents.

Another embodiment of the invention related to method of administering the composition to a subject. The composition may administered through oral, intravenous, intramuscular or subcutaneous routes.

Yet another embodiment, the said composition is administered at a dose level ranging between about 1 and 100 mg per kg body weight/day, preferably in the range of 1 to 25 mg/kg body weight.

Yet another embodiment, the said composition may be administered for period ranging between at least four weeks and up to twelve weeks, and in case of relapse it can again can be administered to the subject without any toxicity.

Yet another embodiment, the said composition inhibits the growth of leukemic cell types K562. and Molt-4.

Yet another embodiment, the said composition inhibits the growth of leukemic cell types Molt-4.

Yet another embodiment, the said composition, IC₅₀ value for in vitro activity against K562 cells of concentration of 10⁴/well is up to 27.0 nanomole/ml.

One more embodiment of the invention provides a method of treating a subject suffering from chronic myeloid leukemia where Bcr-Abl kinase is constitutively expressed in animals and humans or any diseases caused by the over expression of Abl type of kinase.

In another embodiment, the IC₅₀ values for chlorogenic acid and sodium chlorogenate on K562 cells is 27.0 and 13.5 μm/10⁴ cells respectively (FIGS. 2C & 2D). Another embodiment, the acute toxicity of sodium chlorogenate in mouse model, wherein the compound is non-toxic up to a dose level of 2 gm/kg body weight in oral route.

The invention is described in details with reference to the examples given below which are provided to illustrate the invention and therefore, should not be construed to limit the scope of the invention.

### BRIEF DESCRIPTION OF THE ACCOMPANYING DRAWINGS

FIG. 1. Structure of sodium chlorogenate, one of the salts of chlorogenic acid.

FIG. 2. Purification of chlorogenic acid from Piper betel leaves and its effects on cell lines and Ph^{+/-} CML patients' PBMC in vitro. The flow diagram of purification is shown. The structure of peak 09 isolated from fraction E by HPLC was deduced as chlorogenic acid by spectroscopic methods (IR, NMR, ¹³CNMR, FABMS). Cell count assays were performed by plating cells in the presence of regular growth medium with or without indicated amount of extract, FIG. 2(a), fraction, FIG. 2(b), purified compound, FIG. 2(c), and its sodium salt, FIG. 2 (d). Each day, viable cells were counted as assessed by exclusion of trypan blue, FIG. 2(e). Viability of PBMC obtained from three Ph⁺ CML patients and one Ph⁻ CML patient after treatment with Na-Chl (67.5 nmole/10⁵ cells), FIG. 2(f), Morphological changes of K562 cells after treatment with NaChl (67.5 nmole/10⁵ cells) for 6 h (phase contrast micrographs, ×400).

FIG. 3. Sodium chlorogenate induces apoptosis in Ph⁺ CML cell line and CML patient's PBMC.

FIG. 3(a). Cells were left untreated (NT) or incubated with NaChl (67.5 nmole/10⁵ cells) for 6 h and processed for flow cytometry after staining with annexin V-FITC and PI. Viable cells are in the lower left quadrant. Apoptotic cells stained by annexin V are in the lower right quadrant. Late stage apoptotic cells stained by both annexin V and PI are in the upper right quadrant.

FIG. 3(b). Treatment with NaChl results in early cell cycle arrest followed by apoptosis in Ph⁺ cells. Cells were cultured in the presence or absence of NaChl. After 1 or 2 days in culture, cells were collected, permeabilized, and stained with PI for DNA content analysis. Gates were set to assess the percentages of dead (<2 n DNA, M1). G0/G1 (2 n DNA, M2), and S+G2+M (>2 n DNA, M3) cells.

FIG. 3(c). Fluorescence images of K562 and Molt-4 cells after treatment with NaChl followed by staining with annexin V-Allexa™ staining with annexin V are shown in left panels and bright filed phase contrast views are shown in right panels.

FIG. 3(d). Treatment with NaChl leads to activation of procaspase-3 in Ph⁺ cells. Cells were left untreated or treated with NaChl (T) for 24 h. Cells were harvested, lysed, equivalent amount of lysates were separated by SDS-PAGE and electro-transferred. The filters were probed with anti-caspase-3 that recognize both procaspase-3 (32 Kda, upper band) and caspase-3 cleavage product (17 Kda, lower band).

FIGS. 4 (a)-(d) Illustrate how sodium chlorogenate inhibits Bcr-Abl autophosphorylation in Ph⁺ cells.

FIG. 4(a) Flow cytometric determination of Abl phosphorylation status. Cells were left untreated (NT) or incubated with NaChl (T) for 3 h, permeabilized and stained with rabbit anti-phospho-c-Abl (Tyr245) antibody. Dotted line, staining with normal rabbit IgG; solid line, staining with immune IgG.

FIG. 4(b) Immuno-blot-based determination of Abl phosphorylation status. Cells were harvested, lysed, equivalent amount lysates were separated by SDS-PAGE and electrotransferred. The filters were probed with anti-phospho-c-Abl (Tyr 245) (upper panels) or anti-c-Abl antibody (middle panels). To demonstrate equal protein levels in the samples analysed, anti-actin antibody was also used as loading controls (bottom panes).

FIG. 4(c) Flow cytometric determination of Abl expression status. Cells were permeabilized and stained with rabbit anti-c-Abl antibody. Dotted line, staining with normal rabbit IgG; solid line, staining with immune IgG.

FIG. 4(d) Structures of the complexes of the inactive (Panel-A & B) form of Abl tyrosine kinase with gleevec (A) and Chl (B) and active (Panel-C & D) form of the kinase with PD173955 (C) and Chl (D). Structures of complexes of the inactive form with gleevec (A) and active form with PD173955 (C) were obtained by x-ray crystallography. Structures of complexes of the inactive form with Chl (B) and active form with Chl (D) were modeled using those x-ray structures. Ribbons show the binding pocket of the kinase; yellow lines are the parts of activation loop, which differs in inactive (A&B) and active (C&D) conformations. Envelopes around the inhibitor molecules are their Connolly surfaces.

FIG. 5. K562 cells embedded in Matrigel were staged in nude mice until tumors reached 200-300 mm³. PBS (●), NaChl (25 mg/kg, □; 100 mg/kg, Δ; 150 mg/kg, [symbol] ) were administered intraperitoneally once a day for ten days.

FIG. 6. NaChl induces apoptosis of ST1571-sensitive (∘) and -resistant K562 cells (K562-R, ▪). K562 and K562-R cells were incubated with graded concentrations of ST1571 (A) or NaChl (B) for 48 h, proliferation was then determined by [³H] thymidine uptake.

### EXAMPLES

### Example 1

Preparation of Sodium Chlorogenate:

4.7 kg of Piper betel leaves freshly collected, washed with distilled water and then cut into small pieces. Small pieces of leaves were gathered together and mixed with 1.0 litre of distilled water and thoroughly homogenized in a mixture blender. The homogenate was passed through a fine cheesecloth to filter out the large particles and the filtrate was collected. The process was repeated 2-3 times to have maximum yield. The combined filtrate was then centrifuged, the aliquot, a clear solution, was collected and lyophilised to a semi-solid mass, which was about 110 gm. Collected material was examined for biological activity i.e. destruction of CML cells. On observing its positive activity, purification was initiated. 10 gm of above-mentioned material was loaded on Sephadex LH-20 column and chromatographed with water, water-methanol (1:1) and methanol as eluent. Three different fractions thus obtained from three different solvent systems were separately checked for biological activity. The activity was located only on Methanol-water (1:1) and termed as fraction E. Fraction E (0.23 g) was then subjected to preparative HPLC using M-Bonda pak column (19×300 mm) with a solvent system methanol:water:acetic acid (23:76:1), having flow rate of 12 ml/min and detection at 280 nm. A purified compound, chlorogenic acid (4 mg) was isolated from the peak (peak no. 09) having retention time 9.16 min.

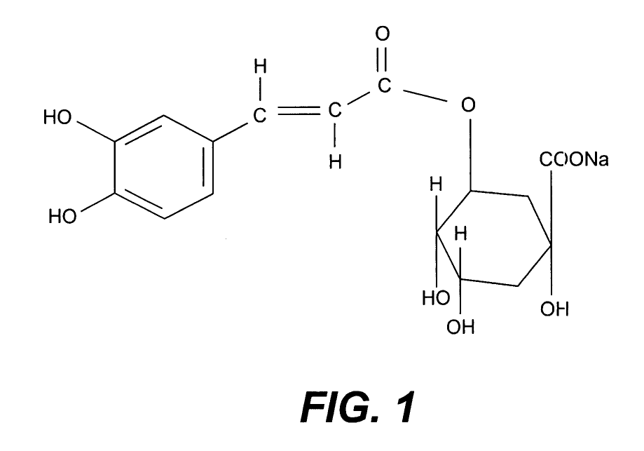

Sodium Chlorogenate (13 mg) was prepared by stirring 10.5 mg of Chlorogenic acid with sodium hydrogen carbonate (3.6 mg) in 2 ml of water and then lyophilised from the resulting solution. It was tested for biological activity. The structure was thus determined as sodium chlorogenate (FIG. 1) IR, NMR, ¹³CNMR and FABMS m/2.

KBr
IR γmax cm⁻¹ : 3398(OH), 1685(CO), 1593, 1527 1449, 1443, 1394, 1279, 1159, 1118 1081, 1038, 975, 916 and 810
¹H-NMR (D₂O) : 7.45(1H), 6.99 (1H), 6.93(1H), 6.77(1H) 6.18(1H), 5.16(1H), 4.11(1H), 3.75(1H) and 2.09-1.85(4H)
¹³C NMR (D₂O) : 181.28, 169.49, 147.69, 146.45, 144.67, 127.14, 123.10, 116.51, 115.40, 114.68, 77.28, 73.33, 71.56, 71.17, 38.88 and 37.72
FABMS m/Z : 355 (M⁺+H) and 377 (M⁺+Na)

### Example 2

The Chlorogenic acid is available in the market in the pure form. The Chlorogenic acid (1 gm) was hand shaken with sodium hydrogen carbonate (0.24 g in 5 ml of water) solution. The solution was lyophilised to pure sodium chlogogenate (1.12 gm) and then was tested for biological activity. Sodium chlorogenate prepared from chlorogenic acid which was either isolated from Piper betel or obtain commercially have similar structure and activity.

### Example 3

Culture of Bcr-Abl positive CML cell line (K562), peripheral blood cells of CML patients, Bcr-Abl-negative ALL cell line (Molt-4) and peripheral blood cells of CML patients. Cell count assays were performed by plating cells in the presence of regular growth medium with or without indicated amount of extract, fraction, purified compound and its sodium salt. Each day, viable cells were counted as assessed by exclusion of trypan blue.

### Example 4

Morphology analysis of Bcr-Abl positive CML cell line K562 by phase contrast microscopy. Cells were left untreated (NT) or treated with NaChl (Nachl; 67.5 nmole/10⁵ cells) and viewed under phase contrast microscope (magnification×400).

### Example 5

Measurement of apoptosis by flow cytometry. Cells were left untreated or treated with NaChl (67.5 nmole/10⁵ cells) for 6 h. After washing, cells were stained with fluorescein isothyocyanate (FITC) conjugated Annexin V and propidium iodide (PI) and analysed in a flow cytometer (FACS Calibur, Beckton Dickinson, USA).

### Example 6

Confocal microscopy. K562 and Molt-4 cells were treated with NaChl followed by staining with Annexin-V-Allexa™ as described in example 5, and allowed to adhere onto poly-L-lysine-coated coverslips for 10 min. Representative fields of cells were analysed with a Leica TCS SP2 confocal laser scanning microscope (Heidelberg, Germany).

### Example 7

DNA cell cycle analysis. Cells were cultured with NaChl as described in example 5. After 1 or 2 days culture, cells were collected, permeabilized and stained with PI for DNA cell cycle analysis.

### Example 8

Immunoblot assay. Cells were harvested, lysed, equivalent amount of lysates were separated by SDS-PAGE and electro-transferred. The filters were probed with anti-caspase-3 antibody (B.D. Pharmingen), anti-c-Abl antibody or anti-phospho-c-Abl antibody (Cell Signaling Technology).

### Example 9

Flow cytometric determination of Abl phosphorylation or Abl expression status. Cells were permeabelysed, stained with rabbit anti-phospho-c-Abl antibody, anti-c-Abl antibody or control rabbit antibody and analysed in a flow cytometer.

### Example 10

Structures of the complexes of Chl with the inactive and active forms of the kinase were modeled using the InsightII 98.0 (Accelrys). Models of the complexes were built using two recently determined structures of two complexes of the enzyme with two important drug molecules, which have some structural and functional similarities with Chl. The structure of Chl was built and optimized by repeated minimization and dynamic simulations. The initial structure of a complex was built by superposing a functional group of Chl with a similar group of the experimental structure of the drug molecule. It was optimized by energy minimization (100 steps each of steepest descent and conjugate gradient methods) using cff91 force field. Then dynamics was run for 1000 steps of one fempto second each after 100 steps of equilibration with a conformational sampling of 1 in 10 steps at 300° K. At the end of the simulation the conformation with lowest potential energy was picked for the next cycle of simulation. This combination of minimization and dynamics were repeated until a satisfactory conformation was obtained. A series of optimizations were done with varying initial conditions in the cavity of the binding pocket. Position constraints were applied to the atoms which were more than 10 Å away during energy minimization and molecular dynamics.

### Example 11

In vivo studies on K562 xenografts: K562 cells were suspended to 5×10⁷ cells/ml in matrigel (BD Biosciences, Mountain View, Calif.; 1 volume of cells with 1 volume of cold Matrigel). Nude female mice 6 to 7 weeks of age were given injections of 0.2 ml of this suspension. Animals were left untreated until K562 xenografts reached 200-300 mm³. Sodium chlorogenate (NaChl) at varying doses (25- 150 mg/kg) was administered intraperitoneally once a day for 10 days (5 mice per group). Phosphate buffered saline (PBS, 0.2 ml per mouse) was used as a control. Animal studies were conducted under an approved institutional Animal Care and Use Committee protocol.

### Example 12

K562 cells were incubated with increasing concentrations of STI571 (received as gift in the form of Gleevec from a CML patient receiving Gleevec therapy), and surviving cells were collected by centrifugation and treated with 2-fold higher concentrations of STI571 (Donato, N.J. et al. Blood, 101:690-698, 2003).

K562 and STI571-resistant K562 cells (K562-R) were incubated with graded concentrations of STI571 or NaChl for 48 hours, proliferation was then determined by [³H]thymidine uptake.

### Example 13

Effect of chlorogenic acid on the cytokine production by normal human peripheral blood mononuclear cells (HPBMC). HPBMC were separated from heparinised whole blood by Ficoll/Hypaque density gradient centrifugation, washed and incubated with chlorogenic acid (25.0 μg/ml) in the presence or absence of phytohemoagglutinin (PHA, 2.5 μg/ml) and lipopolysaccharide (LPS, 1.0 μg/ml) for 18 hrs. Supernatants were collected and quantitated for IL-2, IFN- [symbol] ), IL-6 by commercial ELISA.

### Observed Results

Results of Examples 3 & 4:

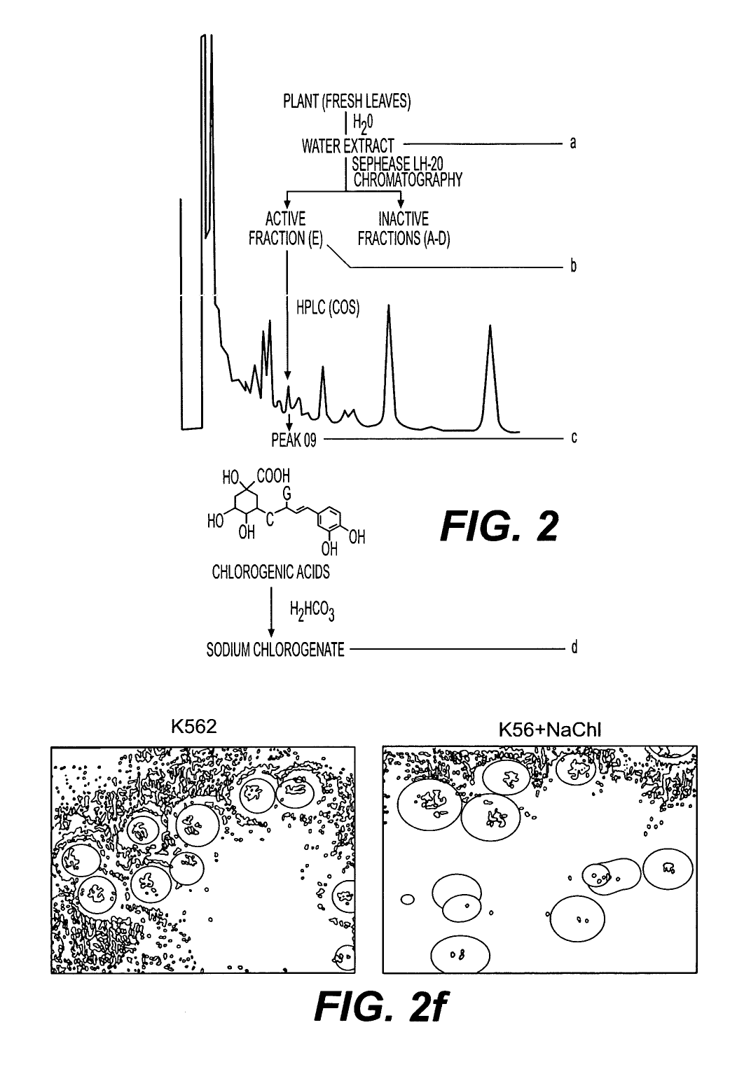

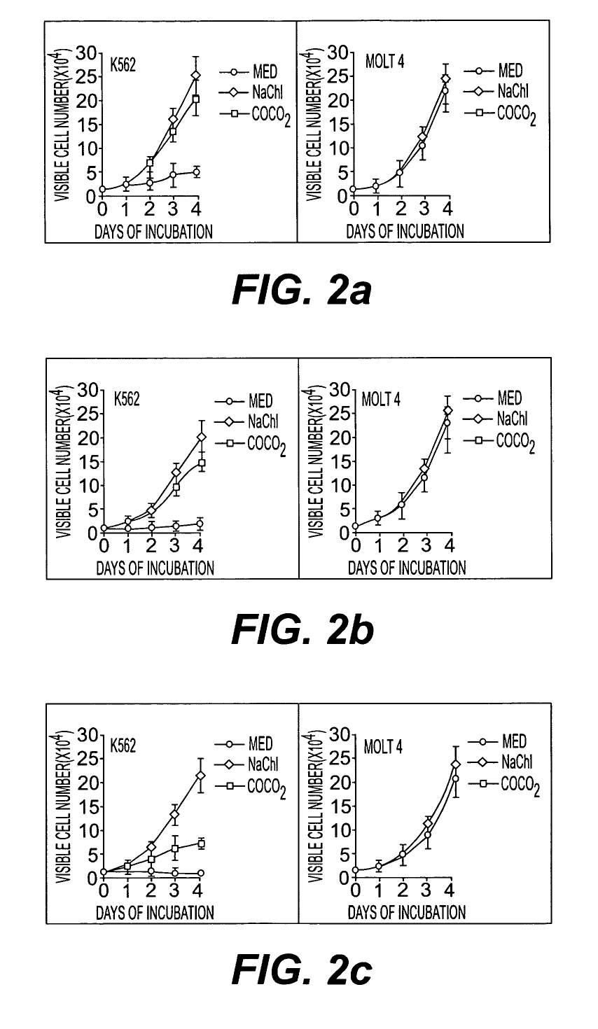

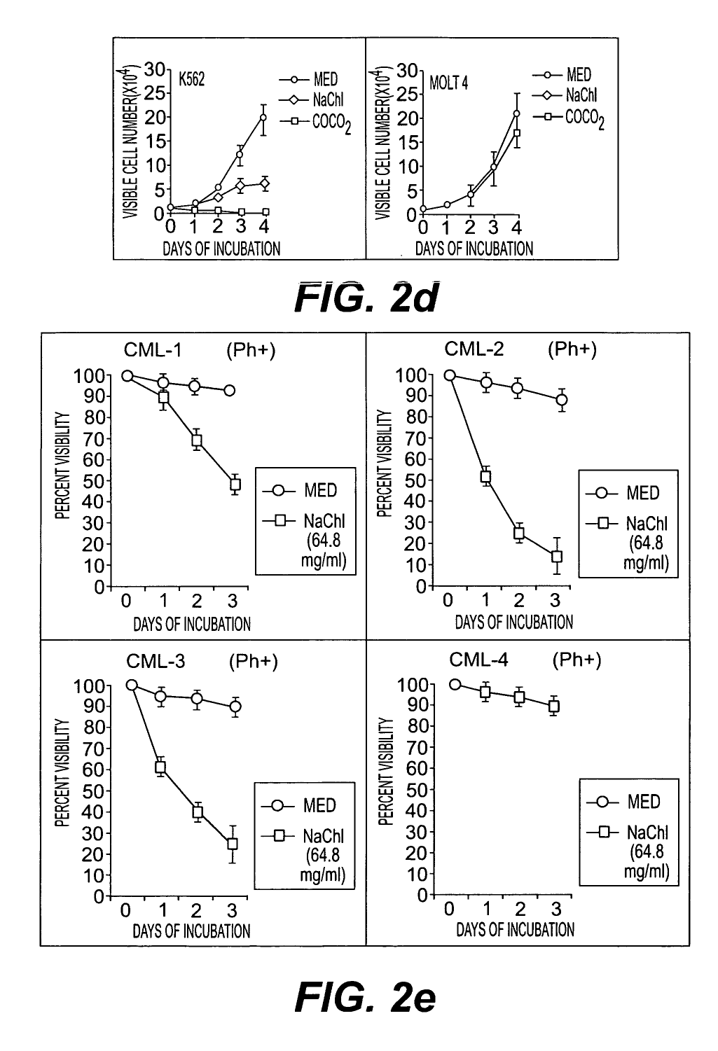

Water extract of Piper betel leaves killed K562 cells in a dose dependent manner (FIG. 2a). The extract had no appreciable effect on AAL cell line Molt-4. Fraction E induced killing activity of K562 cells was restricted to peak 09 (FIG. 2b) which was subsequently identified as chlorogenic acid (FIG. 2c) and showed higher killing activity of K562 cells compare to crude extract or fraction E. Interestingly, sodium chlorogenate (NaChl) is two-fold more potent than chlorogenic acid in killing K562 cells (FIG. 2d). NaChl is also active in killing Philadelphia chromosome (Bcr-Abl) positive CML patients peripheral blood mononuclear cells (PBMC) (FIG. 2e). NaChl has no effect on Bcr-Abl negative CML patients PBMC (FIG. 2e). Phase contrast microscopy indicates that NaChl induces morphological changes (nuclear condensation) in K562 cells, sign of apoptosis (FIG. 2f).

The IC50 values for chlorogenic acid and sodium chlorogenate on K562 cells is 27.0 and 13.5 μm/10⁴ cells respectively, as shown in FIGS. 2C & 2D. With respect to acute toxicity of sodium chlorogenate in mouse model, the compound is non-toxic upto the dose level of 2 gm/kg body weight in oral route.

Results of Examples 5 to 9:

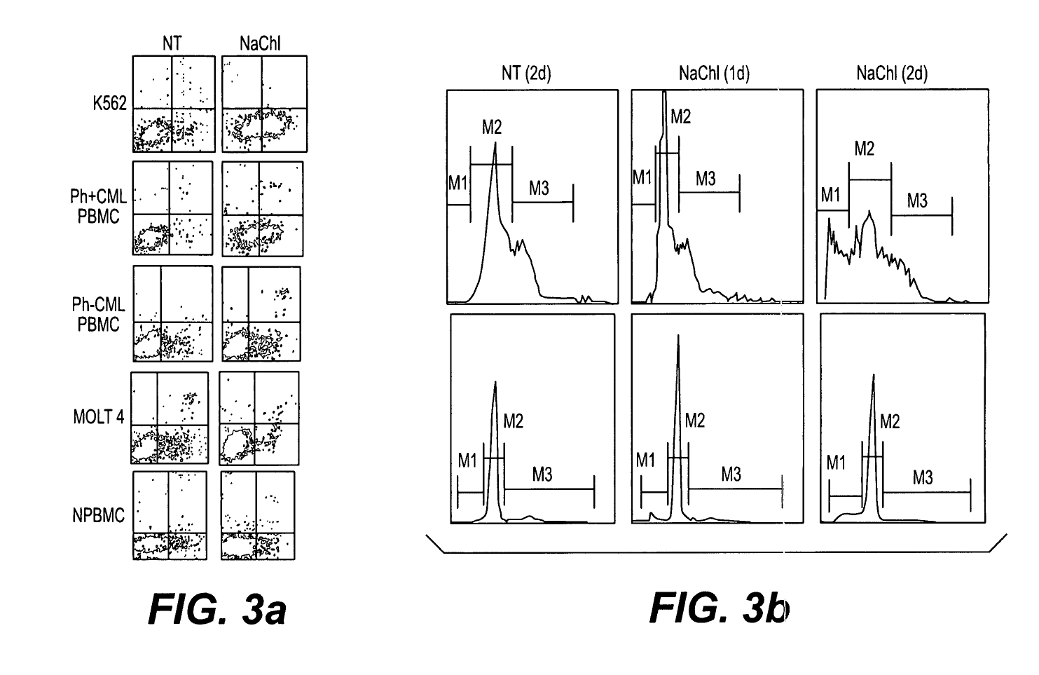

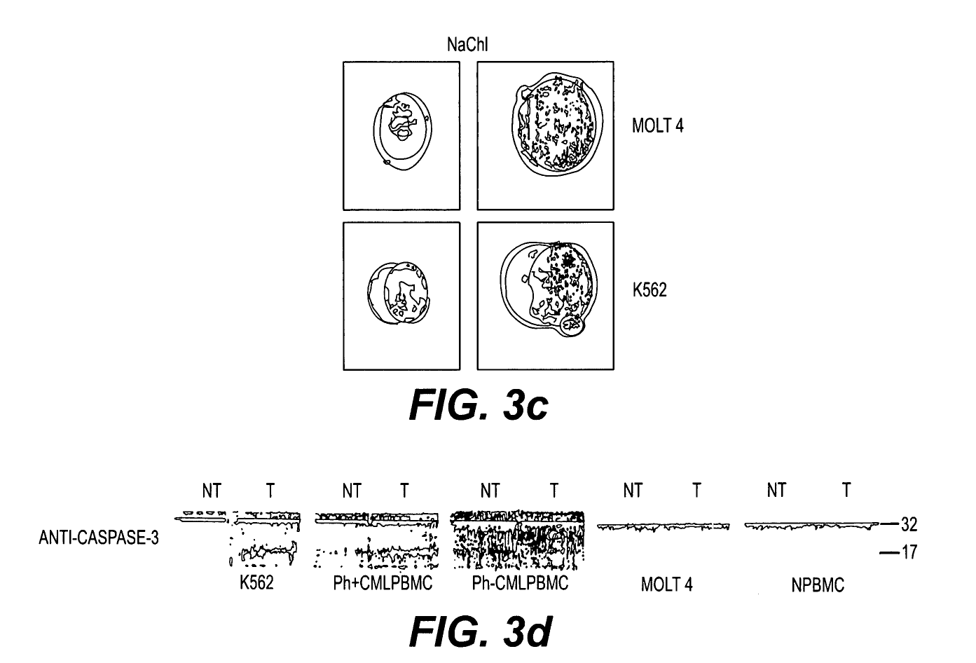

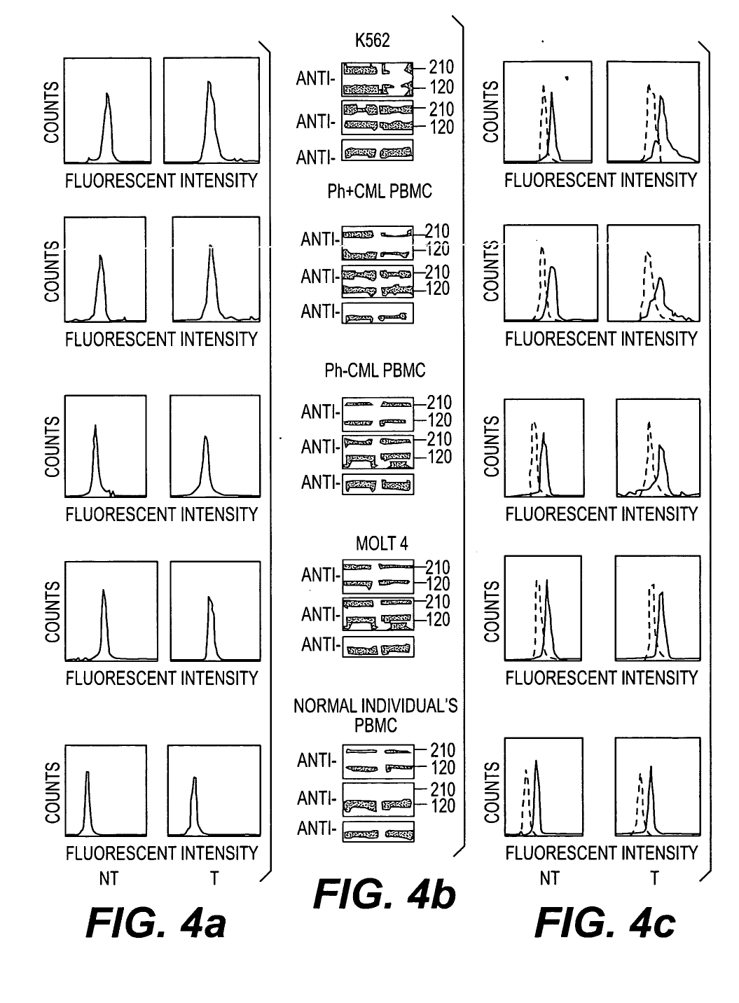

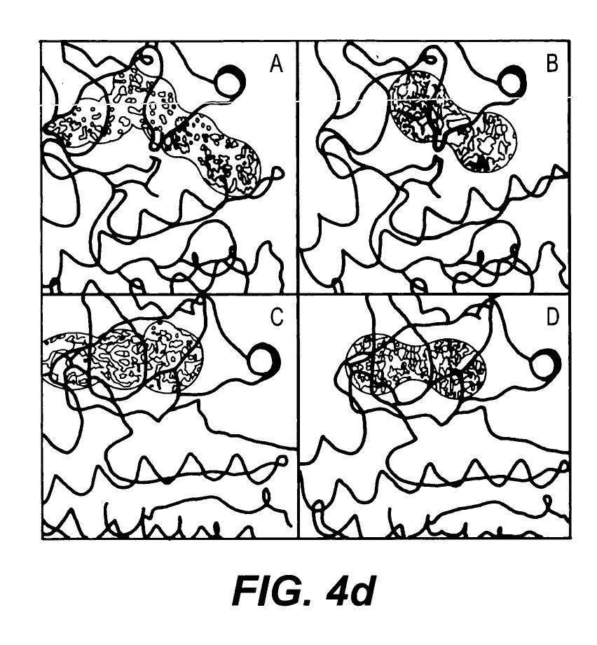

Treatment with NaChl did not induce apoptosis in Molt-4 cells, normal PBMC or PBMC of Bcr-Abl negative CML patients. In contrast, the same treatment cause an increase in apoptosis in Bcr-Abl positive K562 cells and PBMC of Bcr-Abl positive CML patients (FIG. 3a). DNA cell cycle analysis also indicates that NaChl induces cell cycle arrest followed by breakdown of DNA in Bcr-Abl positive CML cell line K562 cells and PBMC of Bcr-Abl positive CML patients (FIG. 3b). Confocal microscopy indicating positive Annexin V staining in K562 cells but not in Molt-4 cells after treatment with NaChl is shown in FIG. 3c. Apoptosis in K562 cells, PBMC of Bcr-Abl (Ph) positive CML patients but not in Molt-4 cells or PBMC of Bcr-Abl negative CML patients or of normal donors was further confirm by immunoblot detection of caspase 3 activation (FIG. 3d). NaChl inhibits phosphorilation of Bcr-Abl protein tyrosine kinase without affecting the expression of Abl protein level. This is evident by flow cytometric detection of phospho-c-Abl status (FIG. 4a), and by immunoblot detection (FIG. 4b, upper panels). Expression of Abl protein was analysed also by flow cytometry (FIG. 4c) and immunoblot (FIG. 4b, middle panels). Our finding, therefore, indicates that sodium chlorogenate inhibits phosphorylation of Abl protein tyrosine kinase including Bcr-Abl kinase leading to apoptosis of cells. The IC50 values for chlorogenic acid and sodium chlorogenate on K562 cells is 27.0 and 13.5 μm/10⁴ cells respectively, as shown in FIGS. 2C & 2D. With respect to acute toxicity of sodium chlorogenate in mouse model, the compound is non-toxic up to the dose level of 2 gm/kg body weight in oral route.

Results of Example 10

FIG.-4d shows that chlorogenic acid (Chl, Panel-B) can fit into the binding pocket of Abl kinase in the inactive conformation in a position similar to that of Gleevec (Panel-A) and in the active conformation (Panel-D) similar to that of PD173955 (Panel-C). Empirical energies associated with the docking of the ligand into the binding pockets of the active and inactive conformations of the kinase are negative and comparable to those of other small molecule inhibitors e.g. Gleevec and PD173955 indicating stable complex formation. Binding energies of Chl in charged and neutral forms are different and the magnitude of electrical interactions depends on the electrical state of the molecule unlike the neutral inhibitors. Modeling studies indicate that Chl can bind to both the active and inactive conformations of the kinase like PD173955. Chl forms a number of hydrogen bonds with the surrounding residues as found in the complex of Gleevec while keeping some of the hydrophobic interactions intact. In comparison to PD173955, Chl forms higher number hydrogen bonds while maintaining similar number of hydrophobic contacts. It has been found that the aromatic hydroxyl groups of Chl forms a network of hydrogen bonds in the binding pocket suggesting the importance of these groups in the complex formation.

Results of Examples 11 and 12:

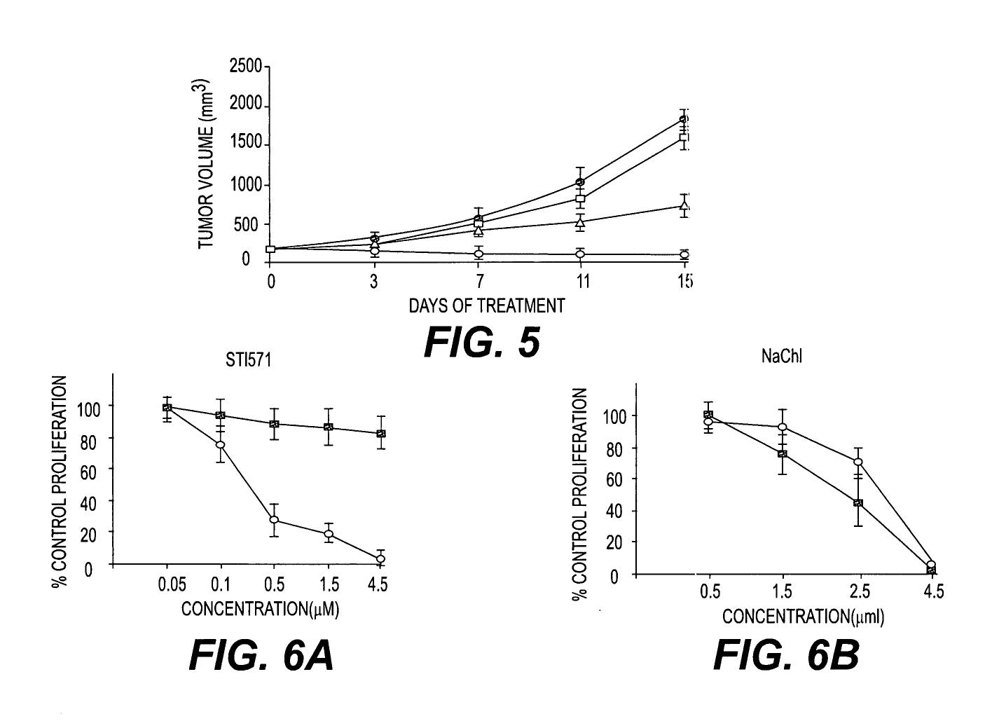

Administration of NaChl in nude mice bearing K562 xenografts reduced the tumor growth in a dose dependent manner (FIG. 5).

NaChl induces apoptosis of ST1571 -sensitive and -resistant K562 cells in vitro (FIG. 6).

Results of Example 13:

Chlorogenic acid inhibits the spontaneous production of IL-2, IFN- [symbol] , and IL-6 by HPBMC (Table II). Production of these cytokines by HPBMC after stimulation with PHA and LPS is also significantly inhibited by chlorogenic acid (Table II).

TABLE II
Chlorogenic acid inhibits the production of Cytokines
by human Peripheral blood mononuclear cells (HPBMC)

| | Concentration of Cytokines | | |
|---|---|---|---|
| | (pg/ml) | | |
| HPBMC treated with | IFN-γ | IL-2 | IL-6 |
| Media alone | 24.5 | 10.0 | 960.9 |
| Chl (25 μg/ml) alone | 19.4 | 6.8 | 781.9 |
| PHA (2.5 μg/ml) + LPS (1.0 μg/ml) | 251.3 | 80.2 | 2797.4 |
| PHA (2.5 μg/ml) + LPS (1.0 μg/ml) + | 13.8 | 2.5 | 1899.0 |
| Chl (25 μg/ml) | | | |

Chl: Chlorogenic acid;
PHA: Phytohemoagglutinin;
LPS: Lipopolysaccharide

1. A pharmaceutical composition useful for treating chronic myeloid leukemia where Bcr-Abl kinase is constitutively expressed in animals and humans, said composition comprising an effective amount of analogs of chlorogenic acid and/or its salts along with pharmaceutically acceptable additives.

2. A composition as claimed in claim 1, wherein said composition is also useful for diseases caused by over expression of Abl type of kinase.

3. The composition as claimed in claim 1, wherein the analogs of chlorogenic is selected from a group consisting of sodium chlorogenate, neochlorogenic acid (5-O-caffeoyl quinic acid), cryptochlorogenic acid (4-O-Caffeoyl quinic acid), 3-O-(3′-methylcaffeoyl) quinic acid and 5-O-(Caffeoyl-4′-methyl) quinic acid.

4. The composition as claimed in claim 1, wherein the analogs of chlorogenic acid are obtained either from natural sources or synthetically prepared.

5. The composition as claimed in claim 1, wherein the salt of chlorogenic acid analog is selected from sodium, potassium and ammonium.

6. The composition as claimed in claim 1 wherein, the additive is selected from a group consisting of nutrients such as proteins, carbohydrates, sugars, talc, magnesium stearate, cellulose, calcium carbonate, starch-gelatin paste and/or pharmaceutically acceptable carriers, excipient, diluents or solvents.

7. The composition as claimed in claim 1 is administered through oral, intravenous, intramuscular or subcutaneous routes.

8. The composition as claimed in claim 1 is administered at a dose level ranging between 1 and 20 mg per kg body weight/day.

9. The composition as claimed in claim 1, which is administered for at least four weeks and up to twelve weeks.

10. The composition as claimed in claim 1, wherein said composition is also useful for relapsed conditions of CML.

11. The composition as claimed in claim 1 wherein, the said composition inhibits the growth of leukemic cell types K562 and Molt-4.

12. The composition as claimed in claim 1, wherein the IC₅₀ value for in vitro activity against K562 cells is up to 27.0 μm/10⁴ of K562 cells.

13. Use of pharmaceutical composition as claimed in claim 1 for the treatment of chronic myeloid leukemia in a subject where Bcr-Abl kinase is constitutively expressed in animals and humans and the analogs of chlorogenic is selected from a group consisting of sodium chlorogenate, neochlorogenic acid (5-O-caffeoyl quinic acid), cryptochlorogenic acid (4-O-Caffeoyl quinic acid), 3-O-(3′-methylcaffeoyl) quinic acid and 5-O-(Caffeoyl-4′-methyl) quinic acid.

14. Use of a pharmaceutical composition for the treatment of chronic myeloid leukemia in a subject where Bcr-Abl kinase is constitutively expressed in animals and humans, said composition comprising an effective amount of analogs or salts of chlorogenic acid represented by Formula 1, as follows, along with pharmaceutically acceptable additives:

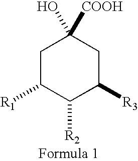

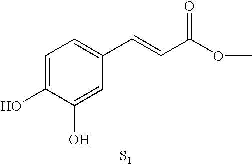

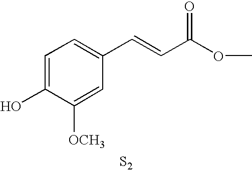

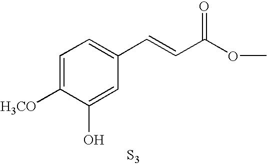

| | Identity of | Identity of | Identity of |
|---|---|---|---|
| Compound | R₁ | R₂ | R₃ |
| Neochlorogenic acid | S₁ | OH | OH |
| Cryptochlorogenic acid | OH | S₁ | OH |
| 3-O-(3′-methyl caffenoyl) quinic acid | OH | OH | S₂ |
| 5-O-(caffenoyl-4′-methyl) quinic acid | S₃ | OH | OH |

15. A use as claimed in claim 14 wherein the subject is selected from a mammal preferably a human being.

16. A use as claimed in claim 14, wherein said composition is also useful for diseases caused by over expression of Abl type of kinase.

17. A use as claimed in claim 14, wherein the salt of chlorogenic acid analog is selected from sodium, potassium and ammonium.

18. A use as claimed in claim 14 wherein, the additive is selected from a group consisting of nutrients such as proteins, carbohydrates, sugars, talc, magnesium stearate, cellulose, calcium carbonate, starch-gelatin paste and/or pharmaceutically acceptable carriers, excipient, diluents or solvents.

19. A use as claimed in claim 14, wherein said composition is administered through oral, intravenous, intramuscular or subcutaneous routes.

20. A use as claimed in claim 14 wherein the said composition is administered at a dose level ranging between 1 and 20 mg per kg body weight/day.

21. A use as claimed in claim 14 wherein the said composition is administered for at least four weeks and up to twelve weeks.

22. A use as claimed in claim 14 wherein, the said composition inhibits the growth of leukemic cell types K562 and Molt-4.

23. A use as claimed in claim 14 wherein the IC₅₀ value for in vitro activity against K562 cells is up to 27.0 μm/10⁴ of K562 cells.

24. A method of treating chronic myeloid leukemia CML where Bcr-Abl kinase is constitutively expressed in animals and humans, said method comprising administering a pharmaceutical composition comprising an effective amount of analogs or salts of chlorogenic acid represented by Formula 1, as follows, along with pharmaceutically acceptable additives:

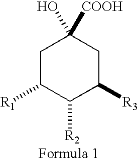

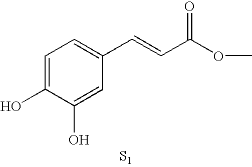

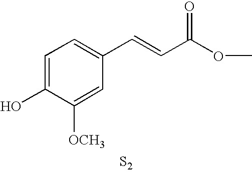

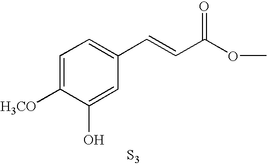

| | Identity of | Identity of | Identity of |
|---|---|---|---|
| Compound | R₁ | R₂ | R₃ |
| Neochlorogenic acid | S₁ | OH | OH |
| Cryptochlorogenic acid | OH | S₁ | OH |
| 3-O-(3′-methyl caffenoyl) quinic acid | OH | OH | S₂ |
| 5-O-(caffenoyl-4′-methyl) quinic acid | S₃ | OH | OH |

25. A method as claimed in claim 24, wherein the said composition is also useful for diseases caused by over expression of Abl type of kinase.

26. The method as claimed in claim 24, wherein the analogs of chlorogenic acid are obtained either from natural sources or synthetically prepared.

27. The method as claimed in claim 24, wherein the salt of chlorogenic acid analog is selected from sodium, potassium and ammonium.

28. The method as claimed in claim 24, wherein, the additive is selected from a group consisting of nutrients such as proteins, carbohydrates, sugars, talc, magnesium stearate, cellulose, calcium carbonate, starch-gelatin paste and/or pharmaceutically acceptable carriers, excipient, diluents or solvents.

29. The method as claimed in claim 24, wherein the said composition is administered through oral, intravenous, intramuscular or subcutaneous routes.

30. The method as claimed in claim 24, wherein the said composition is administered at a dose level ranging between 1 and 20 mg per kg body weight/day.

31. The method as claimed in claim 24, wherein the said composition is administered for at least four weeks and up to twelve weeks.

32. The method as claimed in claim 24, wherein the said composition is also useful for relapsed conditions of CML.

33. The method as claimed in claim 24wherein, the said composition inhibits the growth of leukemic cell types K562 and Molt-4.

34. The method as claimed in claim 24, wherein the IC₅₀ value for in vitro activity against K562 cells is up to 27.0 μm/10⁴ of K562 cells.

35. A pharmaceutical composition useful for treating chronic myeloid leukemia where Bcr-Abl kinase is constitutively expressed in animals and humans, said composition comprising an effective amount of analogs or salts of chlorogenic acid represented by Formula 1, as follows, along with pharmaceutically acceptable additives:

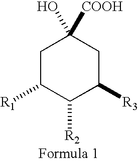

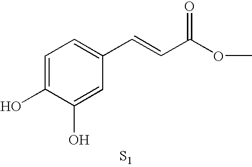

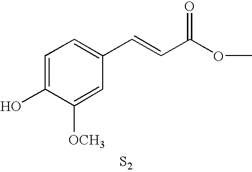

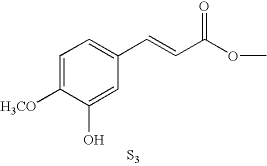

| | Identity of | Identity of | Identity of |
|---|---|---|---|
| Compound | R₁ | R₂ | R₃ |
| Neochlorogenic acid | S₁ | OH | OH |
| Cryptochlorogenic acid | OH | S₁ | OH |
| 3-O-(3′-methyl caffenoyl) quinic acid | OH | OH | S₂ |
| 5-O-(caffenoyl-4′-methyl) quinic acid | S₃ | OH | OH |

36. A composition as claimed in claim 35, wherein said composition is also useful for diseases caused by over expression of Abl type of kinase.

37. The composition as claimed in claim 35, wherein the salt of chlorogenic acid analog is selected from sodium, potassium and ammonium.

38. The composition as claimed in claim 35, wherein the additive is selected from a group consisting of nutrients such as proteins, carbohydrates, sugars, talc, magnesium stearate, cellulose, calcium carbonate, starch-gelatin paste and/or pharmaceutically acceptable carriers, excipient, diluents or solvents.

39. The composition as claimed in claim 35 which is to be administered through oral, intravenous, intramuscular or subcutaneous routes.

40. The composition as claimed in claim 35 which is to be administered at a dose level ranging between 1 and 20 mg per kg body weight/day.

41. The composition as claimed in claim 35, which is to be administered for at least four weeks and up to twelve weeks.

42. The composition as claimed in claim 35, wherein said composition is also useful for relapsed conditions of CML.

43. The composition as claimed in claim 35 wherein, the said composition inhibits the growth of leukemic cell types K562 and Molt-4.

44. The composition as claimed in claim 35, wherein the IC₅₀ value for in vitro activity against K562 cells is up to 27.0 μm/10⁴ of K562 cells.
